SEQUENCE LISTING (12 sequences)

SEQ ID NO: 1 - DNA, 20 bp, Yellow Croaker
cgaaaacatcccgtctgcct

SEQ ID NO: 2 - DNA, 20 bp, Yellow Croaker
gggccaacttcaatgccaaa

SEQ ID NO: 3 - DNA, 23 bp, Yellow Croaker
cagaacaaataaaccatctcgcc

SEQ ID NO: 4 - DNA, 18 bp, Yellow Croaker
atttgaaacagactgggc

SEQ ID NO: 5 - DNA, 22 bp, Yellow Croaker
caggtcatcatcctgaaccacc

SEQ ID NO: 6 - DNA, 22 bp, Yellow Croaker
gagaagctctccacacacatgg

SEQ ID NO: 7 - DNA, 20 bp, Homo Sapien
actggtgagacctgcgtgta

SEQ ID NO: 8 - DNA, 20 bp, Homo Sapien
atccatcggtcatgctctcg

SEQ ID NO: 9 - DNA, 20 bp, Homo Sapien
catggatgaggagactggca

SEQ ID NO: 10 - DNA, 20 bp, Homo Sapien
tgccctcagcaacaagttca

SEQ ID NO: 11 - DNA, 21 bp, Homo Sapien
ccactggaagcaggaatgagt

SEQ ID NO: 12 - DNA, 20 bp, Homo Sapien
tggtgctcaagccacagttg

(19) United States
(12) Patent Application Publication
(10) Pub. No.: US 2024/0093153 A1
(43) Pub. Date: Mar. 21, 2024

(54) METHODS OF MEAT PRODUCTION BY IN VITRO CELL CULTIVATION

(71) Applicant: Avant Meats Company Limited, Hong Kong, S.A.R. (CN)

(72) Inventors: Po San Mario CHIN, Hong Kong (CN); Kai Yie Carrie CHAN, Hong Kong (CN); Chun Hei POON, Hong Kong (CN)

(73) Assignee: Avant Meats Company Limited, Hong Kong, S.A.R. (CN)

(21) Appl. No.: 17/782,148

(22) PCT Filed: Jul. 28, 2020
(86) PCT No.: PCT/IB2020/057119

§ 371 Date: Jun. 2, 2022

Related U.S. Application Data
(60) Provisional application No. 62/942,568, filed on Dec. 2, 2019.

(51) Int. Cl. C12N 5/077; A23L 17/00; C12N 15/113

(52) U.S. Cl. CPC C12N 5/0656; A23L 17/00; C12N 15/113; C12N 2310/141; C12N 2510/00

(57) ABSTRACT

A method for meat production by in vitro cell culture includes isolating tissue from an animal or plant source and making a cell suspension of cells, and growing the cells into a solid or semi-solid structure that mimics an animal organ by growing the cells on a food grade scaffold in a culture medium. Expression of one or more proteins in the growing cells may be increased by altering a level of one or more micro RNAs that regulate expression of the protein. Additionally, the growing cells may be co-cultured with bioengineered cells that secrete growth factors and cytokines that support the growth of the cells in situ. The co-culturing technique reduces or eliminates the need for animal-derived fetal bovine serum in the culture medium.

### SEQUENCE LISTING

The present application incorporates a sequence listing a separate part of the disclosure of the application.

### TECHNICAL FIELD

Embodiments discussed herein generally relate to improved methods for meat production using in vitro cell culture.

### BACKGROUND

Animal meat is high in protein, and supplies all the amino acids needed to build the protein used to support body functions. Meat for consumption is traditionally obtained from animals or fish that are reared on farms. However, agriculture and aquaculture for producing animal meat require a large amount of energy and resources, and have a high carbon footprint. Meat produced by agriculture or aquaculture may pose a public health risk as the production processes may expose the meat to diseases, pollutants, and toxins. A number of concerns such as a growing population, increasing demand for meat, environmental concerns, limited land and water resources, biodiversity loss, and the negative perception associated with animal slaughter have led scientists to develop techniques to produce meat by alternative processes.

In vitro meat production is the process by which muscle tissue or organ tissue from animals are grown in laboratories using cell culture techniques to manufacture meat and meat products. As used herein, in vitro meat and meat products includes animal protein products as well as non-meat products including soluble forms and solid forms. While still in an early stage of development, in vitro meat and meat products may offer a number of advantages over traditional meat production such as health and environmental advantages, and benefits to animal welfare. It is a next generation and emerging technology that operates as part of a wider field of cellular agriculture, or the production of agricultural products from cell cultures.

Cells for the production of in vitro meat may be cells (e.g., muscle cells, somatic cells, stem cells, etc.) taken from animal biopsies, which may then be grown separately from the animal in culture media in a bioreactor or other type of sterile environment. The cells may grow into a semi-solid or solid form mimicking an animal organ by attaching to an edible three-dimensional scaffold that is placed in the bioreactor. The starter cells may be primary cells directly obtained from the animal's tissues, or continuous cell lines. If grown under the right conditions in appropriate culture media, primary cells will grow and proliferate, but only a finite number of times that is related to the telomere length at the end of the cell's DNA. Continuous cell lines, on the other hand, can be cultured in vitro over an extended period. Cell biology research has established procedures on how to convert primary cells into immortal continuous cell lines. Primary cells may be transformed into continuous cell lines using viral oncogenes, chemical treatments, or overexpression of telomerase reverse transcriptase to prevent the telomeres from shortening.

The culture media may contain components necessary for cell proliferation such as amino acids, salts, vitamins, growth factors, and buffering systems to control pH. Current methods add fetal bovine serum (FBS) to the media prior to use as it provides vital macromolecules, growth factors, and immune molecules. However, FBS is derived from unborn calves and, therefore, is incompatible with the objective of being free from animal products. Growing the cells in an animal component-free medium is an important factor considered by scientists involved in in vitro meat production research. Some growth factors may be derived from human sources.

Current in vitro meat production covers most commodity meat types, such as, cell-based beef, pork and poultry meats. However, these types of meats have a complex tissue organization involving multiple cell types that are difficult and costly to produce using current biomedical technology techniques. There is also a lack of non-GM methods to increase the protein level and biomass yield in meat produced by cell culture techniques. Furthermore, as explained above, current cell culture technologies may rely on animal components (e.g., FBS) as a nutrient source, as well as expensive non-food grade growth factors.

The embodiments of the present disclosure apply methods for in vitro meat production for human consumption that provide a solution to the above challenges.

### SUMMARY

According to one embodiment of the present disclosure, a method for meat production by in vitro cell culture includes isolating tissue from an animal or plant source and making a cell suspension of cells. The method further includes growing the cells on a food grade scaffold in a culture medium such that the cells grow into a solid or semi-solid structure that mimics an animal organ. Additionally, the method further includes increasing expression of a protein in the growing cells by altering a level of one or more micro RNAs that regulate the expression of the protein.

According to another embodiment of the present disclosure, a method for meat production by in vitro cell culture includes isolating tissue from a plant or animal source and making a cell suspension of cells, and growing the cells on a food grade scaffold in a culture medium such that the cells grow into a solid or semi-solid structure that mimics an animal organ. The method further includes co-culturing the cells with bioengineered cells that secrete nutrients, growth factors, and cytokines that support the growth of the cells.

Embodiments disclosed herein apply methods for in vitro meat production for human consumption that provide a solution to the above challenges.

### BRIEF DESCRIPTION OF THE DRAWINGS

The disclosure may be better understood by reference to the detailed description when considered in connection with the accompanying drawings. The components in the figures are not necessarily to scale, emphasis instead being placed upon illustrating the principles of the disclosure.

FIG. 1 is a flowchart of a method for meat production by in vitro cell culture, according to one embodiment of the present disclosure.

FIG. 2 is a schematic representation of a method for post-transcriptional enhancement of protein expression, according to one embodiment of the present disclosure.

FIG. 3 is a schematic representation of a method for post-transcriptional enhancement of collagen, type 1, alpha 1 (COL1A1) expression, according to one embodiment of the present disclosure.

FIG. 4 is a schematic representation of a method for post-transcriptional enhancement of collagen, type 1, alpha 2 (COL1A2) expression, according to one embodiment of the present disclosure.

FIG. 5 is a schematic or conceptual cross-sectional view of a bioreactor used for in vitro meat production having a solid phase support, according to one embodiment of the present disclosure.

FIG. 6 is a schematic or conceptual cross-sectional view of a bioreactor similar to FIG. 5 but having a second solid phase, according to one embodiment of the present disclosure.

FIG. 7 is a graphical representation of data regarding mRNA expression of COL1A1 in YCB cells at 24 hour post-transfection by negative control miRNA, miRNA 132 inhibitor (miR-132 inhibitor), miRNA 133 (miR-133 inhibitor), and miRNA 21 mimic (miR-21 mimic) according to one embodiment.

FIG. 8 is a graphical representation of data regarding mRNA expression of COL1A2 in YCB cells at 24 hour post-transfection by negative control miRNA, miR-132 inhibitor miR-133 inhibitor, and miR-21 mimic according to one embodiment.

FIG. 9A is a chromatogram of expression of COL1A1 in YCB cells at 72 hour post-transfection by miR-21 mimic. FIG. 9B is a graphical representation of data regarding expression of COL1A1 in YCB cells at 72 hour post-transfection by miR-21 mimic according to one embodiment.

FIG. 10A is a chromatogram of expression of CTGF in YCB cells at 72 hour post-transfection by negative control miRNA, miR-21 mimic, miR-132 inhibitor, and miR-133 inhibitor. FIG. 10B is a graphical representation of data regarding expression of CTGF in YCB cells at 72 hour post-transfection by negative control miRNA, miR-21 mimic, miR-132 inhibitor, and miR-133 inhibitor according to one embodiment.

FIG. 11 is a graphical representation of mRNA Expression of COL1A1 in 293 cells at 24 hour post-transfection by negative control miRNA and miR-21 mimic. *p<0.05 versus control miRNA according to one embodiment.

### DETAILED DESCRIPTION

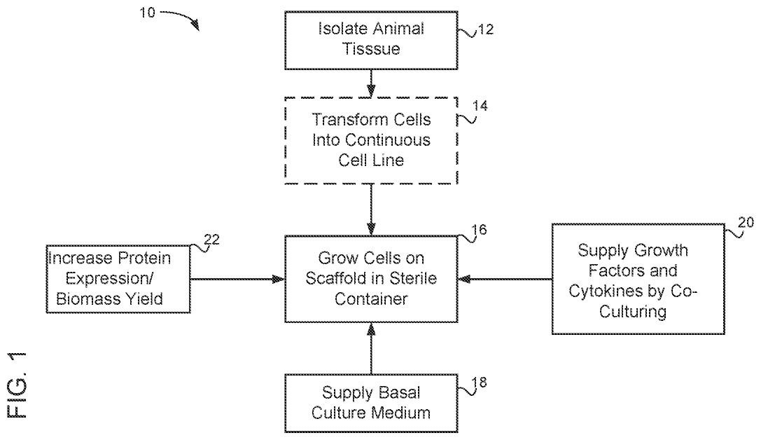

Referring now to the drawings, and with specific reference to FIG. 1, a method 10 for in vitro meat production is shown. As used herein, “in vitro meat production” refers to a cell-based meat production process or cell-based agriculture process in which tissues from animals and/or plants are grown in laboratories using cell culture techniques to manufacture meat and meat products. At a block 12, tissue from an animal or a plant is isolated. In one embodiment, the tissue is derived from bony fish of the class Osteichthyes including saltwater fish such as a grouper, sea bass, or a yellow cocker. In other embodiments, other types of animal tissue, such as cow tissue, may be isolated. In some embodiments, the block 12 may involve collecting organ tissue, such as a swim bladder, from a fish and making a cell suspension. Although the following description primarily describes tissues derived from fish sources, it will be understood that the concepts may be applied to tissues derived from other types of animal sources and/or plant sources to provide other types of in vitro meat and/or animal protein products, and vegetarian meat and/or protein products.

Many of the isolated cells are adult cells, and can be made to proliferate continuously using various established methods in medical research (block 14). For example, specific genes, such as Yamanaka factors, may be used to reprogram the adult cells into stem cells, such as induced pluripotent stem cells (iPSCs). Alternatively, the isolated adult cells may be transformed into continuous cell lines by telomerase reverse transcriptase over expression. In other embodiments, other types of cells may be isolated such as adult stem cells and embryonic stem cells. In this regard, it will be understood that the methods of the present disclosure includes all sources of cell lines.

At a next block 16, the cells are grown into a solid or semi-solid structure mimicking an animal organ, such as a fish organ, by attaching/adhering to a food grade biocompatible scaffold in sterile chamber or container, such as a bioreactor. The sterile chamber or container may be temperature controlled, and may have inlets and outlets for introducing and removing substances such as chemicals, nutrients, and cells. The food grade biocompatible scaffold becomes part of the final edible product, and is made of plant-based or fungi-based materials such as, but not limited to, agarose, alginate, chitosan, mycelium, and konjac glucomannan. Alginate is a biopolymer naturally derived from brown algae and is biocompatible. In addition, plant-based chitosan from fungi has antibacterial properties. In some embodiments, the block 16 is carried out in the absence of antibiotics or antimicrobial compounds in the sterile container. A block 18 involves supplying the culture medium to the bioreactor to support cell survival and growth. The culture medium may be a buffered solution containing components such as, but not limited to, inorganic salts (e.g., calcium chloride (CaCl₂), potassium chloride (KCl), sodium chloride (NaCl), sodium bicarbonate (NaHCO₃), sodium dihydrogen phosphate (NaH₂PO₄), magnesium sulfate (MgSO₄), etc.), amino acids, vitamins (e.g., thiamine, riboflavin, folic acid, etc.), and other components such as glucose, β-mercaptoethanol, ethylenediaminetetraacetic acid (EDTA), and sodium pyruvate. Non-limiting examples of growth media include, but are not limited to, Leibovitz's L-15 medium, Eagle's Minimum Essential Media (MEM), Medium 199, Dulbecco's Modified Eagle Medium (DMEM), Ham's F12 Nutrient Mix, Ham's F10 Nutrient Mix, MacCoy's 5A Medium, Glasgow Modified Eagle Medium (GMEM), Iscove's Modified Dulbecco's Medium, and RPMI 1640.

According to a block 20, food grade growth factors and cytokines are introduced into the culture medium in the bioreactor to support cell growth and proliferation. The growth factors and cytokines may include, but are not limited to, insulin growth factor 1 (IGF-1), insulin, interleukin 6 (IL-6), interleukin 6 receptor (IL-6R), interleukin 11 (IL-11), fibroblast growth factor (FGF), epidermal growth factor (EGF), and transferrin. The block 20 may involve co-culturing bioengineered cells with the isolated cells in the absence of fetal bovine serum (FBS). The bioengineered cells are engineered to secrete the above growth factors and cytokines, and supply these biomolecules to the isolated cells as needed for growth and proliferation. As used herein, “bioengineered” cells are not equivalent to genetically-modified cells. The bioengineered cells have a specific gene that overexpresses one or more specific proteins. The bioengineered cells may be fish cells, or other types of animal cells, such as cow cells. The bioengineered cells are not present in the final meat product. As non-limiting examples, bioengineered fish cells may be co-cultured with isolated fish cells, or bioengineered cow cells may be co-cultured with isolated cow cells. The co-culturing method of the present disclosure eliminates the need for animal-derived fetal bovine serum (FBS) in the culture medium. Furthermore, the co-culturing method provides a continuous supply of food grade specific growth factors and cytokines to the growing cells in situ, and simplifies and reduces the cost of the production process. However, in other embodiments, FBS, other serum or proteins from recombinant sources may be used to supply growth factors, cytokines, and other nutrients to support cell growth during the block 16.

Additionally, according to a block 22, protein expression in the cells is increased to increase the biomass yield in the resulting meat product. As used herein, “biomass yield” refers to the amount of digestible material (e.g. proteins) in the resulting meat product that is available for energy production upon consumption. More specifically, the block 22 involves increasing protein expression by altering micro RNA levels in the cells, with the manipulation of the cells being carried out prior to culturing. Micro RNAs are endogenous, short, non-encoding single stranded RNA sequences involved in regulating post-transcriptional gene expression. The block 22 involves increasing the amount of up-regulating micro RNAs that increase protein expression by promoting messenger RNA (mRNA) translation, and/or decreasing the amount of down-regulating micro RNAs that decrease protein expression by suppressing mRNA translation. The micro RNA levels may be increased or decreased by introducing micro RNAs, micro RNA mimics, or micro RNA inhibitors into the cells. The micro RNA mimics have the same function as micro RNAs, but may be more stable and efficient in modulating protein expression. In some embodiments, electroporation may be used to introduce episomal vectors into the cells that carry instructions to express specific micro RNAs. Alternatively or in combination with this, adeno-associated virus may be used as a vehicle carrying episomal instructions to express specific micro RNAs. Decreasing the amount of targeted down-regulating micro RNAs may be achieved by introducing inhibitors for the targeted micro RNAs into the cells by transfection. It is noted here that the methods of increasing protein expression/biomass yield according to the present disclosure is carried out without modifying the genome of the cells.

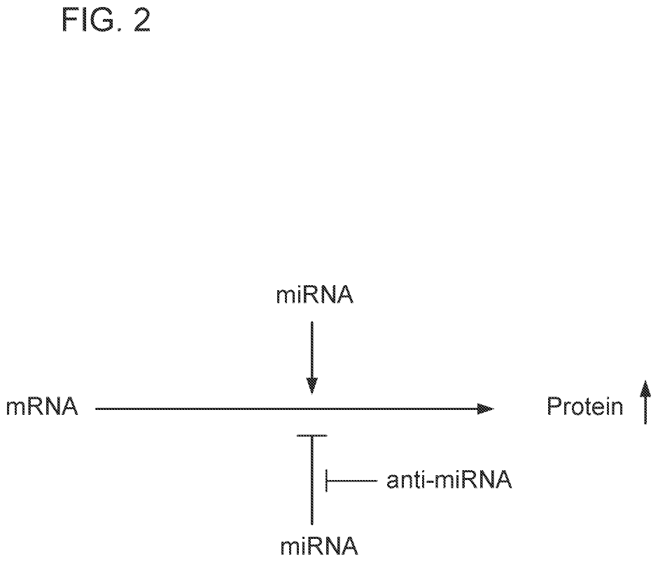

Turning to FIG. 2, a method for post-transcriptional enhancement of protein expression in the cell lines is schematically depicted. One or more up-regulating micro RNAs (miRNAs) may be increased to increase mRNA translation and protein production of selected proteins. Alternatively or in combination with this, one or more down-regulating miRNAs may be blocked with inhibitors (anti-miRNAs) to increase mRNA translation and protein production of selected proteins.

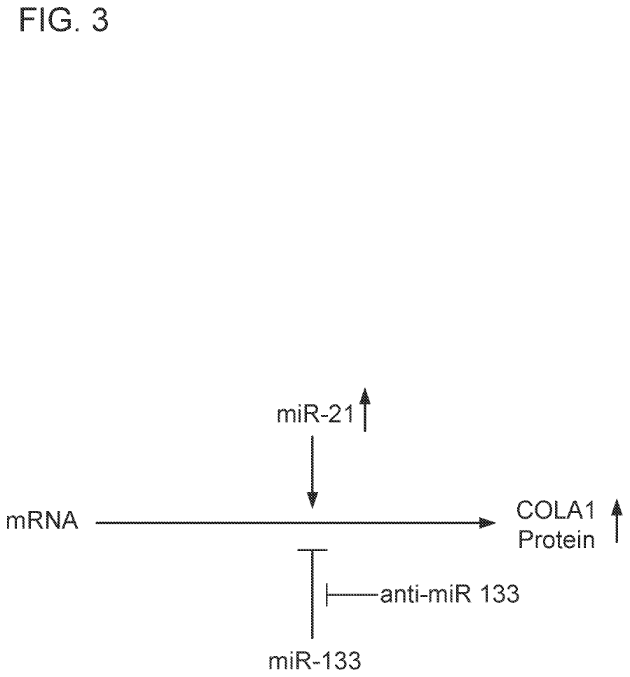

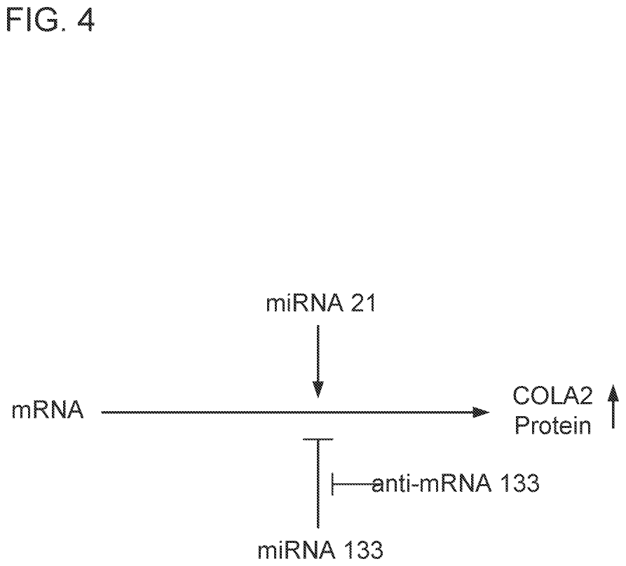

Fish swim bladder primarily includes fibroblasts and collagen protein. Collagen type 1 (collagen I) is a dominant protein in fish swim bladder, and increased expression of collagen I in cultured fish swim bladder cells may increase biomass yield. Collagen I in fish swim bladder cells includes collagen, type 1, alpha 1 (COL1A1) and collagen, type 1, alpha 2 (COL1A2). COL1A1 and COL1A2 expression is increased by up-regulating miRNA 21 (miR-21), such that increasing levels of miR-21 increases COL1A1 and COL1A2 production in fish swim bladder cells. Additionally, COL1A1 and COL1A2 expression is decreased by down-regulating miRNA 133 (miR-133), such that decreasing levels of miR-133 or blocking the action of miR-133 increases COL1A1 and COL1A2 production in fish swim bladder cells. FIGS. 3-4 show increasing COL1A1 (FIG. 3) and COL1A2 (FIG. 4) production by increasing miR-21 levels and by blocking the action of miR-133 with the use of inhibitors (anti-miR 133). Increased COL1A1 and COL1A2 production results in increased biomass yield in the resulting meat product. Similar strategies may be applied to increase relevant protein levels in other types of animal cells.

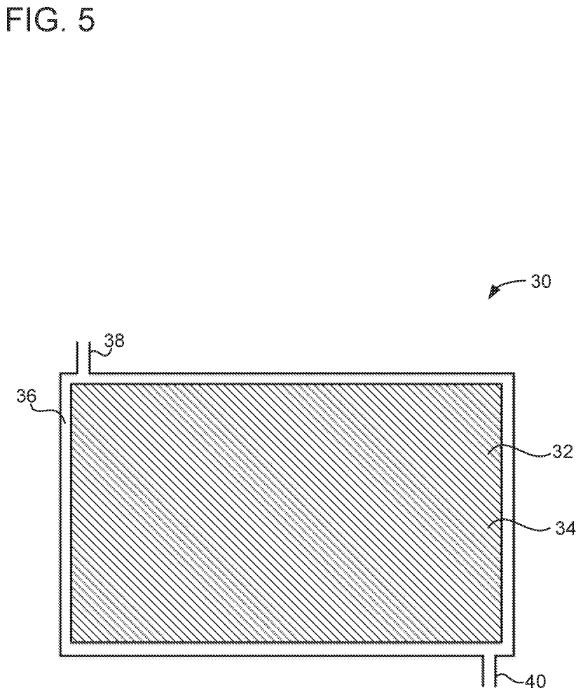

Turning to FIG. 5, an exemplary bioreactor 30 used for culturing the isolated cells is shown. The cells attach to and grow on a solid phase support 32 provided by a food grade scaffold 34 which is held in a sterile chamber 36 in the bioreactor 30. The scaffold 34 may dictate the shape of the meat product. The food grade scaffold 34 is made of plant-based or fungi-based materials such as, but not limited to, agarose, alginate, chitosan, mycelium, and konjac glucomannan. The solid phase support 32 may be porous so that the cells may attach to and grow on inner surfaces of the support 32. The culture medium supplying nutrients to the cells is introduced into the bioreactor 30 through an inlet 38, and is emptied from the bioreactor 30 through an outlet 40.

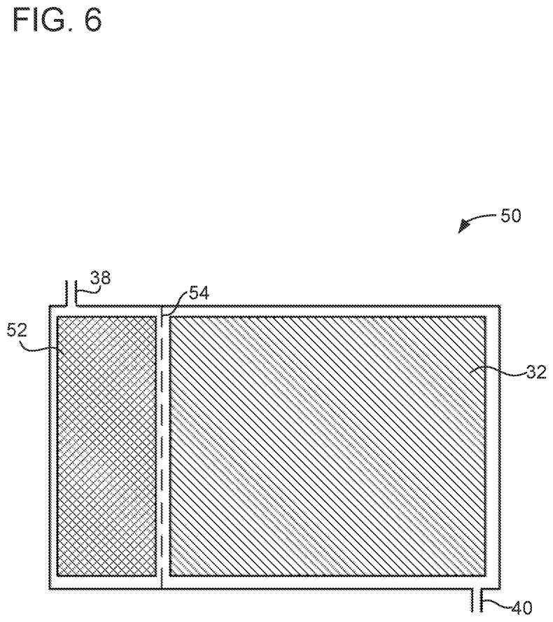

FIG. 6 shows a bioreactor 50 similar to the bioreactor 30 of FIG. 5, but further includes a second solid phase 52 separated from the solid phase support 32 by a fine mesh 54. The second solid phase 52 may contain or support the bioengineered cells that secrete nutrients, growth factors, and cytokines for the cells growing on the solid phase support 32 in situ, and may physically separate the bioengineered cells from the cells on the solid phase support 32. The second solid phase 52 is made of plant-based materials, similar to the solid phase support 32. The mesh 54 is permeable to nutrients, growth factors, and cytokines, but is impermeable to cells. The bioreactor 50 of FIG. 6 allows the co-culturing of the bioengineered cells with the growing cells. In some embodiments, the bioreactors 30 and 50 of FIGS. 5 and 6 may be arranged in tandem. In other embodiments, several of the bioreactors 30, several of the bioreactors 50, or mixtures of the bioreactors 30 and 50 may be arranged in series for scaling up the process. The bioreactor 30 may be used mainly for biomass production, whereas the bioreactor 50 may be used for providing nutrients, growth factors, and cytokines to the growing cells.

The in vitro meat production method of the present disclosure provides meat products with simple tissue organization of one cell type. The meat product with one cell type is easier to make, develop, and commercialize compared other cultured meats having multiple cell types. Alternative embodiments of the present disclosure provide meat products with multiple cell types. Furthermore, Applicant has discovered a strategy to increase biomass/protein production by altering micro RNA levels or activity in the growing cells. In one example, two key micro RNAs (miR-21 and miR-133) are targeted to increase the levels of the dominant protein (collagen I) found in fish swim bladder cells. As far as Applicant is aware, alteration of micro RNA levels or activity to achieve an increased protein/biomass yield in cultured meat products has not been used by others in the field of cultured meat development. Targeting micro RNAs for increased protein production may cause less stress to the cells than known knock-in or knock-out methods. Bio-engineered cells are co-cultured with the growing animal cells to supply the growing fish cells with food grade growth factors and cytokines for cell growth and proliferation in situ, reducing or eliminating the need for animal-derived FBS in the culture medium. The co-culturing technique simplifies the production process and reduces production costs.

Furthermore, the nutrients of the cultivated meat product may be customized to generate a healthier food product. For example, the cultured meat product may be customized according to diet recommendations from a dietician to from a personal genomic test. Healthy nutrients such as high-density cholesterol, polyunsaturated fatty acids, and monounsaturated fatty acids in the meat product may be enriched by culturing the cells in specific conditions. Alternatively, or in combination with this, nutrients known to be damaging to health such as low density cholesterol and saturated fatty acids may be reduced by culturing the cells in specific conditions. Micronutrients, such as vitamins and minerals, may also be enhanced. Nutrient customization of the cultivated meat products may be achieved in various ways such as, but not limited to, 1) tailoring the nutrients fed to the growing cells during cell culture, and/or 2) controlling the proportions of layering scaffolds with different cells.

The production the cultivated food product is under a clean, sterile and highly controlled process. Thus, undesirable degradation by microorganisms such as bacterial or fungi of the nutrients in the food product is minimized. Undesirable taste and smell from the breakdown of nutrients by microorganisms is also minimized. This property of cultivated food enables new uses in cooking and help creates novel recipes. One such application of cultivated food is cultivated fish maw derived from fish swim bladders. Traditional fish maw has an undesirable fishy taste and smell due to the degradation of amine by bacteria in the production process. This undesirable property limits the food ingredient to savory dishes served hot or warm. Cultivated fish maw produced from cell culture technology does not have an undesirable fishy taste and smell. In addition to hot and savory dishes, cultivated fish maw can be used in sweet dishes, as a desert or in a ready-to-eat format served at chilled or at ambient temperature.

### Example 1

### Improved Expression of Protein Using miRNA Mimic and Inhibitors

The Example, according to one embodiment, may set forth a strategy for improve expression of collagen I αI (COL1A1) and collagen I α2 (COL1A2) using miRNA and inhibitors.

As used herein, the term “Inhibitors” may refer to miRNA inhibitors are small, single-stranded RNA molecules designed to specifically bind to and inhibit endogenous miRNA molecules and enable miRNA functional analysis by down-regulation of miRNA activity.

### Materials and Methods

### Reagents

The following reagents were obtained from Thermofisher Scientific: DMEM/F12 with Glutamax™, fetal bovine serum (FBS), trypsin/EDTA, Lipofectamine™ 3000, negative control miRNA (mirVana™ miRNA Mimic, Negative Control #1; Thermofisher Scientific, catalog number #4464058), miRNA 21 mimic (miRBase Accession Number: M10033728; SEQ ID NO: 1; miR-21 mimic), miRNA 132 inhibitor (miRBase Accession Number: MI0000449; SEQ ID NO: 2; miR-132 inhibitor), miRNA 133 inhibitor (miRBase Accession Number: MI0000822; SEQ ID NO: 3; miR-133 inhibitor), RNAlater™ solution, Purelink™ RNA Minikit, Purelink™ DNase Set, and PowerUp™ SYBR™ Green Mastermix. M-MuLVRT (RNase H-) cDNA synthesis kit was purchased from ExCell Bio.

As used herein, “miRNA 21 mimic” has the same RNA sequence as “miRNA 21”.

### Cells

The yellow croaker swim bladder (YCB) cell line was developed in-house. 293 cells were obtained from ATCC. Both cell lines were maintained in complete medium (DMEM/F12, about 2-20% FBS, preferably 10% FBS) inside a humidified incubator (about 2-10% CO₂, preferably 5% CO₂; about 90-98% air, preferably 95% air; about 24-37° C., preferably 34° C.). They were routinely subcultured at a split ratio of 1:2 to 1:10. The split ratio is preferably 1:4.

### Transfection

Cells were transfected by negative control miRNA, miR-21 mimic, miR-132 inhibitor, miR-133 inhibitor with Lipofectamine™ 3000 according to the manufacturer's (Themofisher Scientific) protocol. Cells were detached (when cell confluence ˜80%) by trypsin-EDTA. They were seeded at about 1-10×10⁵/24 well, preferably about 5×10⁵/24 well (YCB cells) or about 1-10×10⁵/24 well, preferably about 6.5××10⁵/24 well (293 cells) in about 0.2-1.0 ml, preferably 0.5 ml, complete medium. Immediately afterwards, about 0.01-0.10 nmol, preferably about 0.06 nmol, miRNA was mixed with about 0.5-5 μl, preferably about 1.5 μl, Lipofectamine™ 3000 reagent in about 50-300 μl, preferably about 100 μl, DMEM/F12. The mixture was incubated at room temperature (about 20° C.-26° C., preferably at about 24° C.) for about 5-30 minutes, preferably about 10 minutes, for complex formation and added dropwise to different areas of the well to achieve a final miRNA concentration of 50-300 μM, preferably 100 μM. Cells were returned to the incubator and analyzed for target gene expression between about 18-36 hour, preferably at about 24 hour.

Total RNA extraction and reverse transcription

At about 18-36 hour, preferably at about 24 hour, post-transfection, the medium was removed, RNAlater™ solution was added to wells to preserve the RNA, and the plates were stored at about 4-10° C., preferably at about 4° C. To extract total RNA, the RNAlater™ solution was removed and RNA was purified by Purelink™ RNA Mini Kit based on the manufacturer's (Themofisher Scientific) protocol. An on-column DNase treatment was performed by Purelink™ DNase Set. The concentration of RNA in the samples were measured by Nanodrop™ 2000c. About 0.5-5 μg RNA, preferably about 2 μg RNA (293 cells) or about 0.5 μg RNA (YCB cells), were reverse transcribed to cDNA using M-MuLVRT (RNase H-) cDNA synthesis kit and oligo dT primer according to the manufacture's (Excell Bio) protocol.

### Real-Time PCR

The expression of COL1A1 and COL1A2 were analyzed by the Roche LC480 real-time PCR system (Roche Diagnostics). cDNA samples were mixed with about 0.1-0.8 μM, preferably about 0.4 μM, gene specific primers (Table 1) and PowerUp™ SYBR™ Green Mastermix. The amplification protocol was as follows: (a) about 45-55° C. for about 1-10 minutes, preferably about 50° C. for about 2 minutes; (b) about 90-98° C. for about 1-5 minutes, preferably about 95° C. for about 2 minutes; (c) about 35-45 cycles, preferably 40 cycles (each cycle may include the following steps: (i) about 90-98° C. for about 10-30 seconds, preferably 95° C. for 15 seconds; (ii) about 50-60° C. for about 10-30 seconds, preferably 55° C. for 15 seconds; and (iii) about 66-74° C. for about 15-60 s, preferably 72° C. for 30 seconds; (iv) melting curve analysis (including the following steps: (1) about 90-98° C. for about 10-30 seconds, preferably about 95° C. for about 15 seconds; (2) about 55-65° C. for about 0.5-3 minutes, preferably about 60° C. for about 1 minute; (3) ramp rate: about 0.05-0.3° C./s until reaching about 90-98° C., preferably about 0.15° C./second until reaching about 95° C.; and (4) capturing signal during ramping). The validity of the PCR was verified by (a) amplificon showing a single peak after melting curve analysis, (b) a single band of correct size was observed when the PCR product after analyzed by gel electrophoresis, (c) amplification efficiency=about 90-110%; Error<about 0.01-0.1. Target gene expression in each sample was determined by the Pfaffl method and was normalized by expression of the housekeeping gene EF1α. The relative gene expression in sample A relative to sample B is governed by equation (1).

$$\frac{{( 1 + amplification efficiency of target gene )}^{[ C t ( A ) - C t ( B ) ]}}{{( 1 + amplification efficiency of EF 1 α )}^{[ C t ( A ) - C t ( B ) ]}} \qquad ( 1 )$$

TABLE 1
Primers used for real-time
PCR in this example

| Gene of | | Primer | |
|---|---|---|---|
| interest | Species | sequence | Amplicon |
| Collagen | Fish | F: 5′-CGAAAACAT | 85 bp |
| Type I | | CCCGTCTGCCT-3′ | |
| α1 (COL1A1) | | R: 5′-GGGCCAACT | |
| | | TCAATGCCAAA-3′ | |
| Collagen | | F: 5′-CAGAACAAA | 107 bp |
| Type I | | TAAACCATCTCGCC-3′ | |
| α2 (COL1A2) | | R: 5′-ATTTGAAAC | |
| | | AGACTGGGC-3′ | |
| Eukaryotic | | F: 5′-CAGGTCATC | 221 bp |
| translation | | ATCCTGAACCACC-3′ | |
| elongation | | R: 5′-GAGAAGCTC | |
| factor 1 | | TCCACACACATGG-3′ | |
| α1 | | | |
| Collagen | Human | F: 5′-ACTGGTGAG | 118 bp |
| Type I | | ACCTGCGTGTA-3′ | |
| α1 | | R: 5′-ATCCATCGG | |
| (COL1A1) | | TCATGCTCTCG-3′ | |
| Collagen | | F: 5′-CATGGATGA | 80 bp |
| Type I | | GGAGACTGGCA-3′ | |
| α2 (COL1A2) | | R: 5′-TGCCCTCAG | |
| | | CAACAAGTTCA-3′ | |
| Eukaryotic | | F: 5′-CCACTGGAA | 100 bp |
| translation | | GCAGGAATGAGT-3′ | |
| elongation | | R: 5′-TGGTGCTCA | |
| factor 1 | | AGCCACAGTTG-3′ | |
| α1 | | | |

### Statistical Analysis

All results are represented as mean±SEM. Statistical analyses were conducted for by Graphpad Prism 5.0. For the gene expression data of YCB cells, data were analyzed by one-way ANOVA followed by, if significant, Tukey post hoc test. For data on 293 cells, data were analyzed by Student's t-test. Results were considered statistically different if p<0.05.

### Results

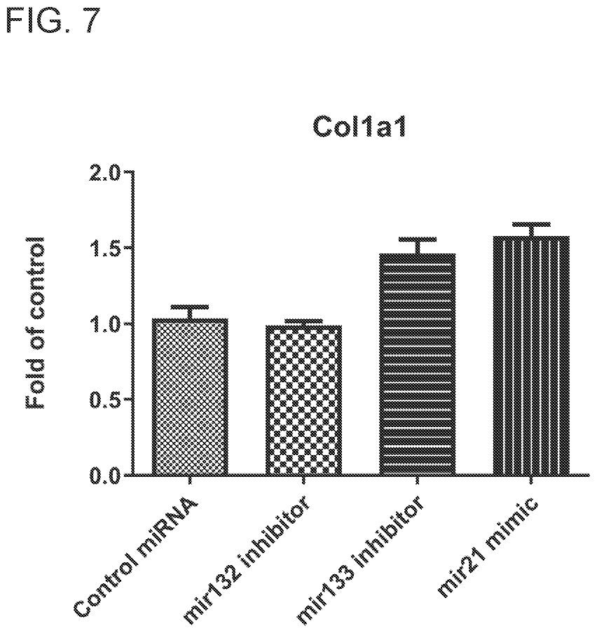

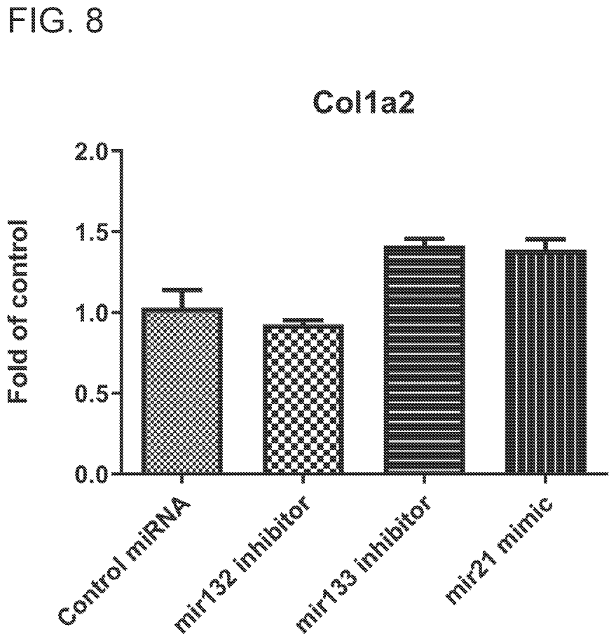

In this example, we transfected YCB cells with negative control miRNA, miR-21 mimic, miR-132 inhibitor, or miR-133 inhibitor. We then quantified two different types of collagen, COL1A1 and COL1A2, mRNA expression at about 24 hour post-transfection by qPCR. We found that as compared to transfection by negative control miRNA, transfection by miR-21 mimic or miR-133 inhibitor increased COL1A1 (FIG. 7) and COL1A2 (FIG. 8) mRNA expression.

As shown in FIG. 7, at 24 hour post-transfection by miR-21 mimic and miR-133 inhibitor, the mRNA expressions of COL1A1 were about 1.5 fold of control and about 1.5 fold of control respectively. The mRNA expressions of COL1A1 were about 1 fold of control and 1 fold of control respectively at about 24 hour post-transfection by negative control miRNA and miR-132 inhibitor.

As shown in FIG. 8, at 24 hour post-transfection by miR-21 mimic and miR-133 inhibitor, the mRNA expressions of COL1A2 were about 1.4 fold of control and about 1.4 fold of control respectively. The mRNA expressions of COL1A2 were about 1 fold of control and about 0.9 fold of control respectively at about 24 hour post-transfection by negative control miRNA and miR-132 inhibitor.

Therefore, it shows that transfection of miR-21 mimic/miR-21 or miR-133 inhibitor increased collagen mRNA expression in YCB cells.

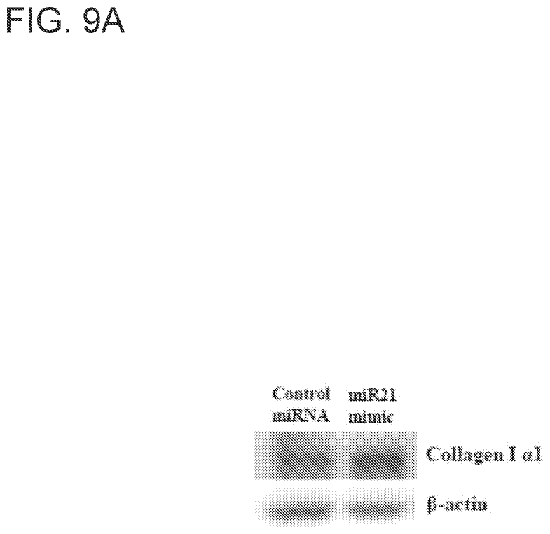

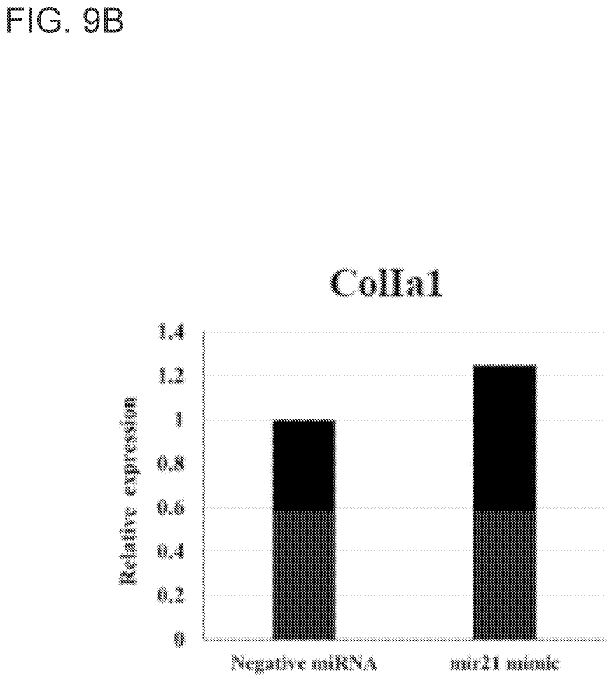

Now turning to FIGS. 9A and 9B, at about 72 hour post-transfection by miR-21 mimic, the protein expression of COL1A1 was increased and the protein expression of COL1A1 was about 1.2 relative expression. The expression of COL1A1 was about 1 relative expression at about 72 hour post-transfection by negative control miRNA.

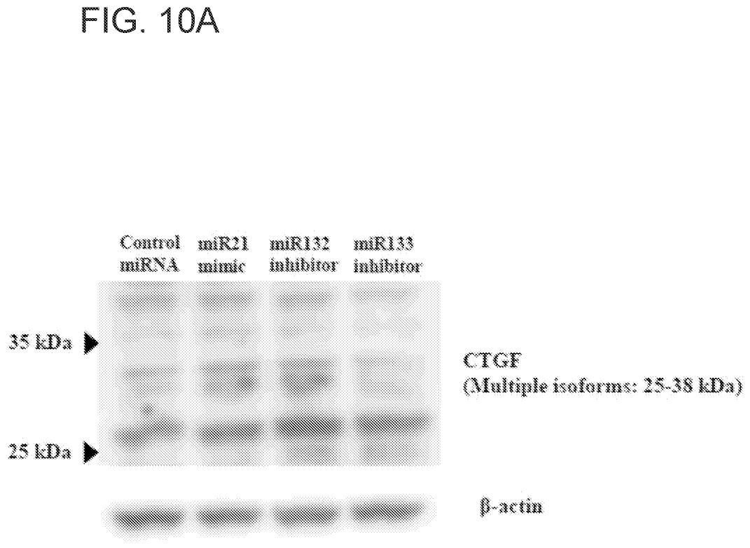

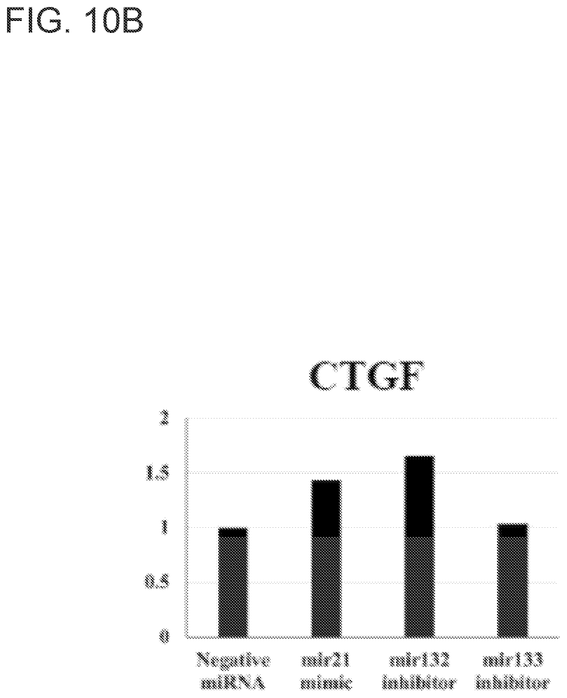

Now turning to FIGS. 10A and 10B, at about 72 hour post-transfection by miR-21 mimic and miR-132 inhibitor, the protein expressions of CTGF were increased and the expressions of COL1A1 were about 1.4 and about 1.6 relative expression respectively. The expressions of CTGF were about 1 relative expression and about 1 relative expression at about 72 hour post-transfection by negative control miRNA and miR-133 inhibitor.

This example shows that miRNAs can control collagen expression in fish swim bladder cells (i.e. YCB cells).

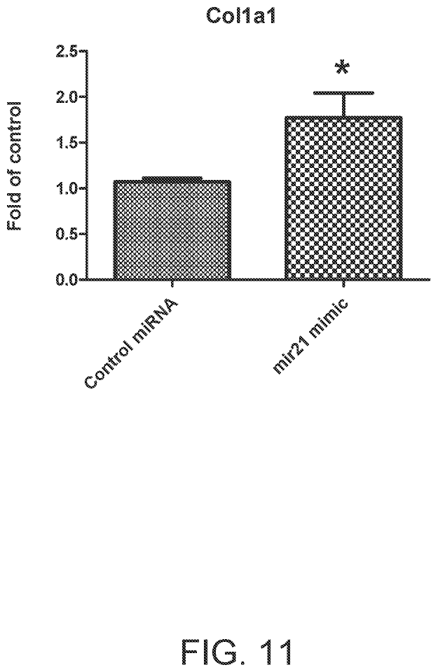

Transfection of miR-21 mimic increased collagen mRNA expression in 293 cells as well. We transfected miR-21 mimic to human 293 cells and analyzed collagen mRNA expression at about 24 hour post-transfection. As shown in FIG. 11, when compared to the negative control miRNA group, miR-21 mimic increased COL1A1 mRNA expression in 293 cells.

As shown in FIG. 11, at about 24 hour post-transfection by miR-21, the mRNA expression of COL1A1 is about 1.6-1.8 fold of control. The mRNA expression of COL1A1 is about 1 fold of control at 24 hour post-transfection by negative control miRNA.

This example shows that the effect of miRNA on collagen expression can be replicated in other cell types.

### CONCLUSION

This example shows that by controlling miRNA at the episomal level, aspects of the invention may increase protein expressions in different cell types. Episomal modulation of specific miRNA can be used to increase the productivity of cells in cultivated meat production.

The above description is illustrative and is not restrictive. Many variations of embodiments may become apparent to those skilled in the art upon review of the disclosure. The scope embodiments should, therefore, be determined not with reference to the above description, but instead should be determined with reference to the pending claims along with their full scope or equivalents.

One or more features from any embodiment may be combined with one or more features of any other embodiment without departing from the scope embodiments. A recitation of “a”, “an” or “the” is intended to mean “one or more” unless specifically indicated to the contrary. Recitation of “and/or” is intended to represent the most inclusive sense of the term unless specifically indicated to the contrary.

While the present disclosure may be embodied in many different forms, the drawings and discussion are presented with the understanding that the present disclosure is an exemplification of the principles of one or more inventions and is not intended to limit any one embodiments to the embodiments illustrated.

The disclosure, in its broader aspects, is therefore not limited to the specific details, representative system and methods, and illustrative examples shown and described above. Various modifications and variations may be made to the above specification without departing from the scope or spirit of the present disclosure, and it is intended that the present disclosure covers all such modifications and variations provided they come within the scope of the following claims and their equivalents.

### Exemplary Protocols

### A. Development of a Fish Bladder Cell Line

1. Obtain a healthy yellow crocker, sea bass or fish of similar category from a local fish market.
2. Keep the fish on ice until cell isolation.
3. Immerse the fish in 10% bleach.
4. Remove swim bladder from the fish under aseptic condition.
5. Wash the organ one or more times in hypochlorous acid.
6. Wash the organ one or more times in antibiotic medium (Leibovitz's L-15 or DMEM or EMEM with 400 IU/ml, penicillin, 400 pg/ml streptomycin).
7. After washing, cut the organ into small pieces (2-3 mm³).
8. Transfer the cut organ to a centrifuge tube containing 0.25% trypsin-EDTA in PBS.
9. Incubate at room temperature with continuous shaking for 1 hour.
10. Filter the supernatant with a 100 μm mesh to remove undigested tissue.
11. Centrifuge the filtrate at 200 g for 5 minutes.
12. Resuspend the cell pellet with complete medium (Leibovitz's L-15 or DMEM or EMEM with 200 IU/ml, penicillin, 200 pg/ml streptomycin, 10% fetal bovine serum).
13. Seed the cell into a T25 flask.
14. Incubate at 24-28° C.
15. Remove cells that are not attached to the tissue culture flask in the next day.
16. Replace half of the medium with fresh medium every 2-3 days.
17. The cells are considered established when a complete monolayer is formed and the established cells are ready for subculture.

### B. Development of a Fish Bladder Cell Line by Tissue Explant

1. Obtain a healthy yellow crocker, sea bass or fish of similar category from a local fish market.
2. Keep the fish on ice until cell isolation.
3. Immerse the fish in 10% bleach.
4. Remove swim bladder from the fish under aseptic condition.
5. Wash the organ one or more times in hypochlorous acid.
6. Wash the organ one or more times in antibiotic medium (Leibovitz's L-15 or DMEM or EMEM with 400 IU/ml, penicillin, 400 pg/ml streptomycin).
7. After washing, cut the organ into small pieces (1-2 mm³).
8. Place organ pieces into a 24 well plate individually containing complete medium (Leibovitz's L-15 or DMEM or EMEM with 200 IU/ml, penicillin, 200 pg/ml streptomycin, 10% fetal bovine serum).
9. Incubate at 24-28° C.
10. Replace half of the medium with fresh medium every 2-3 days without disturbing the tissue explant.
11. Incubate the tissue explant until adherent cells are observed.
12. Remove tissue explant.
13. The cells are considered established when a complete monolayer is formed and the established cells are ready for subculture.

### C. Development of a Fish Muscle Cell Line

1. Obtain a healthy grouper, cod, sole, halibut, flounder or fish of similar category from a local fish market.
2. Keep the fish on ice until cell isolation.
3. Immerse the fish in 10% bleach.
4. Remove muscle from the fish under aseptic condition.
5. Wash the tissue one or more times in hypochlorous acid.
6. Wash the tissue one or more times in antibiotic medium (Leibovitz's L-15 or DMEM or EMEM with 400 IU/ml, penicillin, 400 pg/ml streptomycin).
7. After washing, cut the tissue into small pieces (2-3 mm³).
8. Transfer the cut tissue to a centrifuge tube containing collagenase and dispase in PBS.
9. Incubate at room temperature with continuous shaking for 1 hour.
10. Filter the supernatant with a 100 pm mesh to remove undigested tissue.
11. Centrifuge the filtrate at 200 g for 5 minutes.
12. Resuspend the cell pellet with complete medium (Leibovitz's L-15 or DMEM or EMEM with 200 IU/ml, penicillin, 200 pg/ml streptomycin, 10% fetal bovine serum).
13. Seed the cell into a T25 flask.
14. Incubate at 24-28° C.
15. Remove cell that are not attached to the tissue culture flask in the next day.
16. Replace half of the medium with fresh medium every 2-3 days.
17. The cells are considered established when a complete monolayer is formed and the established cells are ready for subculture.
   D. Development of a Fish Muscle Cell Line from Tissue Explant
1. Obtain a healthy grouper, cod, sole, halibut, flounder or fish of similar category from a local fish market.
2. Keep the fish on ice until cell isolation.
3. Immerse the fish in 10% bleach.
4. Remove muscle from the fish under aseptic condition.
5. Wash the tissue one or more times in hypochlorous acid.
6. Wash the tissue one or more times in antibiotic medium (Leibovitz's L-15 or DMEM or EMEM with 400 IU/ml, penicillin, 400 pg/ml streptomycin).
7. After washing, cut the muscle into small pieces (1-2 mm³).
8. Place muscle pieces into a 24 well plate individually containing complete medium (Leibovitz's L-15 or DMEM or EMEM with 200 IU/ml, penicillin, 200 pg/ml streptomycin, 10% fetal bovine serum).
9. Incubate at 24-28° C.
10. Replace half of the medium with fresh medium every 2-3 days without disturbing the tissue explant.
11. Incubate the tissue explant until adherent cells are observed.
12. Remove tissue explant.
13. The cells are considered established when a complete monolayer is formed and the established cells are ready for subculture.

### E. Adult Stem Cell Isolation and Culturing

1. Obtain a healthy grouper, cod, sole, halibut, flounder or fish 6 months or younger of similar category from a local fish market.
2. Keep the fish on ice until cell isolation.
3. Immerse the fish in 10% bleach.
4. Remove muscle from the fish under aseptic conditions.
5. Wash the tissue one or more times in hypochlorous acid.
6. Wash the tissue one or more times in antibiotic medium (Leibovitz's L-15 or DMEM or EMEM with 400 IU/ml, penicillin, 400 pg/ml streptomycin).
7. After washing, cut the tissue into small pieces (2-3 mm³).
8. Transfer the cut tissue to a centrifuge tube containing collagenase and dispase in PBS.
9. Incubate at room temperature with continuous shaking for 1 hour.
10. Filter the supernatant with a 100 pm mesh to remove undigested tissue.
11. Centrifuge the filtrate at 200 g for 5 minutes.
12. Resuspend the cell pellet with complete medium (Leibovitz's L-15 or DMEM or EMEM with 200 IU/ml, penicillin, 200 pg/ml streptomycin, 10% fetal bovine serum, 100 ng/ml basic fibroblast growth factor).
13. Plate the cells on an uncoated plate for 1 hour at 24-28° C.
14. Harvest the supernatant and place on plate coated with laminin, gelatin, Matrigel or similar matrix.
15. Incubate at 24-28° C.
16. After 24 hours, wash away any loosely attached and non-adherent cells.
17. Replace medium every day with complete medium (Leibovitz's L-15 or DMEM or EMEM with 200 IU/ml, penicillin, 200 pg/ml streptomycin, 10% fetal bovine serum, 100 ng/ml basic fibroblast growth factor).
   F. Generating and Culturing iPSC
1. 2-4 days before transfection, plate cells in complete medium (L15 with 10% FBS) in a tissue culture flask. Cells should be approximately 75-90% confluent on the day of transfection (Day 0).
2. Aspirate the medium from gelatin coated 6-well plates and replace with 2 mL of fresh complete medium per well. Place the coated plates at 37° C. until ready for use.
3. Thaw the Epi5™ vectors at 37° C. and place them on wet ice until ready for use. Before use, briefly centrifuge the thawed vectors to collect them at the bottom of the tube.
4. Wash the cells in PBS.
5. Add 3 mL of 0.05% Trypsin/EDTA to the culture flask containing the cells.
6. Incubate the flask at room temperature for 3 minutes.
7. Add 5-8 mL of complete medium to each flask. Carefully transfer cells into an empty, sterile 15 mL conical tube.
8. Check the viability by trypan blue dye exclusion cell viability assay
9. Centrifuge the cells at 200 g for 2 min.
10. Carefully aspirate most of the supernatant and resuspend with complete medium.
11. Seed cells on gelatin coated dishes plate 50,000 to 100,000 cells per well into a 6-well plate at 30-60% confluence in 2 mL complete medium and Incubate overnight at 24-28° C.
12. Prewarm Opti-MEM/Reduced-Serum Medium to room temperature and prepare Tube A and Tube B as described below.
13. Add 1.2 μL each of the two Epi5™ Reprogramming Vector mixes (2.4 μL total) to 118 μL Opti-MEM medium in a 1.5 mL microcentrifuge tube labeled Tube A. Add 4.8 μL of P3000™ Reagent and mix well.
14. Dilute 3.6 μL Lipofectamine 3000 reagent in 121 μL prewarmed Opti-MEM medium in a 1.5 mL microcentrifuge tube labeled Tube B.
15. To prepare a transfection master mix, add the contents of Tube A to Tube B and mix well.
16. Incubate the transfection master mix for 5 minutes at room temperature.
17. Mix one more time and add the entire 250 μL of transfection master mix to each well.
18. Incubate overnight at 24-28° C.
19. 24 hours post transfection, aspirate the medium from the plates. Add 2 mL N2B27 Medium (L15 with IX N-2 supplement, IX B27 supplement, 100 ng/mL bFGF to each well.
20. Change the N2B27 Medium every day for a total of 14 days by replacing the spent medium with 2 mL N2B27 Medium.
21. Aspirate the spent N2B27 Medium on Day 14 and replace it with complete medium. Resume medium changes every day at 2 mL per well.
22. Observe the plates every other day under a microscope for the emergence of cell clumps, indicative of transformed cells. Within 15 to 21 days post transfection, the iPSC colonies will grow to an appropriate size for transfer.
23. Colonies are distinct by Day 21 and can be picked for further culture and expansion.

### G. Method for Subculturing Cells

1. Remove and discard culture medium.
2. Briefly rinse the cell PBS to remove all traces of serum which contains trypsin inhibitor.
3. Add 2-3 mL of 0.25% Trypsin-EDTA solution to the flask.
4. Incubate at room temperature for 1 min.
5. Add 5-8 mL of complete growth medium.
6. Aspirate cells by gently pipetting.
7. Add appropriate aliquots of the cell suspension to new culture flasks at a subcultivation ratio of 1:2 to 1:3.
8. Incubate at 24-28° C.

### H. Adaption to Suspension Culture

1. Passage monolayer culture at a frequency appropriate for the cell in question by trypsinization.
2. At each passage, wash cell monolayer with PBS and overlay with 0.25% trypsin.
3. Incubate at room temperature for 5 min.
4. Inactivate the enzyme with complete medium.
5. Harvest the cell suspension and check the viability by trypan blue dye exclusion cell viability assay.
6. Seed the cell suspension into another culture flask.
7. Repeat passaging until the viability of the suspended cells is equal or more than 90%.
8. Establish a suspension culture with 50 ml complete medium in a spinner or shaker flask at a cell density of 0.1-0.5 million/ml.
9. Incubate the spinner or shaker flask suspension cultures in a CO₂ incubator under the same conditions of temperature, humidity, and atmosphere optimal for monolayer cultures.
10. Adjust the cell density to 0.1-0.5 million/ml with fresh medium every 2-3 days.
11. Check the viability by trypan blue dye exclusion cell viability assay.
12. Establish multiple parallel cultures at cell density that promote health cell growth.
13. Increase cell density gradually to 1 million/ml using part of the culture.
14. If increasing cell density lead to cell death, discard the high-density culture.
15. Restart high density adaption using cell form step 12.
16. Scale up to a 3 L bioreactor when cells are adapted to grow in suspension.

### I. Adaption to Serum Free Medium (Plant Hydrolysate)

1. Culture cells in DMEM/F12 complete medium (1:1 mixture of DMEM medium and Ham's F12 medium, 2-4 mM glutamine, 10% FBS).
2. Prepare serum free medium (1:1 mixture of DMEM medium and Ham's F12 medium, 2-4 mM glutamine, 20% plant hydrolysate e.g. soy, cottonseed, rapeseed, wheat, yeast or equivalent).
3. When cells reach confluence, replace medium with adaption medium I (40% fresh complete medium, 40% conditioned media from the passage before, 20% serum free medium).
4. Check the viability by trypan blue dye exclusion cell viability assay every 2-3 days.
5. If adaption leads to cell death, discard the culture and repeat step 3.
6. When cells reach confluence, replace medium with adaption medium II (30% fresh complete medium, 30% conditioned media from the cells in step 1, 40% serum free medium).
7. Check the viability by trypan blue dye exclusion cell viability assay every 2-3 days.
8. If adaption leads to cell death, discard the culture and repeat step 6.
9. When cells reach confluence, replace medium with adaption medium III (20% fresh complete medium, 20% conditioned media from the cells in step 1, 60% serum free medium).
10. Check the viability by trypan blue dye exclusion cell viability assay every 2-3 days
11. If adaption leads to cell death, discard the culture and repeat step 9.
12. When cells reach confluence, replace medium with adaption medium IV (10% fresh complete medium, 10% conditioned media from the cells in step 1, 80% serum free medium).
13. Check the viability by trypan blue dye exclusion cell viability assay every 2-3 days
14. If adaption leads to cell death, discard the culture and repeat step 12.
15. When cells reach confluence, replace medium with serum free medium.
16. Check the viability by trypan blue dye exclusion cell viability assay every 2-3 days.
17. If adaption leads to cell death, discard the culture and repeat step 15.
18. The serum free medium usage can be increased more gradually in each step, i.e. an increase of 20% or less in each step.

### J. Adaption to Serum Free Medium (Chemically Defined)

1. Culture cells in DMEM/F12 complete medium (1:1 mixture of DMEM medium and Ham's F12 medium, 2-4 mM glutamine, 10% FBS).
2. Prepare serum free medium (1:1 mixture of DMEM medium and Ham's F12 medium, 2-4 mM glutamine, ascorbic acid 2-phosphate 65-130 ug/ml, NaHCO₃550-1100 ug/ml, sodium selenite 14-28 ng/ml, insulin 19-38 ug/ml, transferrin 11-22 ug/ml, FGF-2 100-200 ng/ml, TGF-beta 2-4 ng/ml).
3. When cells reach confluence, replace medium with adaption medium I (40% fresh complete medium, 40% conditioned media from the passage before, 20% serum free medium).
4. Check the viability by trypan blue dye exclusion cell viability assay every 2-3 days
5. If adaption leads to cell death, discard the culture and repeat step 3
6. When cells reach confluence, replace medium with adaption medium II (30% fresh complete medium, 30% conditioned media from the cells in step 1, 40% serum free medium).
7. Check the viability by trypan blue dye exclusion cell viability assay every 2-3 days..
8. If adaption leads to cell death, discard the culture and repeat step 6
9. When cells reach confluence, replace medium with adaption medium III (20% fresh complete medium, 20% conditioned media from the cells in step 1, 60% serum free medium).
10. Check the viability by trypan blue dye exclusion cell viability assay every 2-3 days.
11. If adaption leads to cell death, discard the culture and repeat step 9.
12. When cells reach confluence, replace medium with adaption medium IV (10% fresh complete medium, 10% conditioned media from the cells in step 1, 80% serum free medium).
13. Check the viability by trypan blue dye exclusion cell viability assay every 2-3 days.
14. If adaption leads to cell death, discard the culture and repeat step 12.
15. When cells reach confluence, replace medium with serum free medium.
16. Check the viability by trypan blue dye exclusion cell viability assay every 2-3 days.
17. If adaption leads to cell death, discard the culture and repeat step 15.
18. The serum free medium usage can be increased more gradually in each step. For example, an increase of 20% or less in each step.

### L. Scaffolding for Cell Culture (Konjac+Gum)

1. Boil water with a few pieces of saffron until the color becomes pale yellow.
2. Remove the saffron and rest the solution until warm.
3. Prepare all dry ingredient
   a. Konjac-0.5-5%, preferably 3%
   b. Baking soda—0.3-3%, preferably 2%
   c. Perfected Xanthan Gum—0.2-2%, preferably 1.5%
4. Measure 100 ml of saffron solution.
5. Add Baking soda, Xanthan Gum sequentially. Stir the mixture well after adding each ingredient.
6. Add Konjac by sprinkling little by little on top of the solution. Keep stirring. The solution should become mushy.
7. Spread the konjac mixture into mold with approximately 1-15 mm thickness.
8. Cover the mold with the lid and rest under room temperature for more than 30 min.
9. Put the mold in 4° C. fridge for 4 hours.
10. Steam the mold under low heat for 40 minutes.
11. Rest the mold under room temperature for 2 hours.
12. Dehydrate the scaffold at 45-55° C. for 15 minutes.

### M. Scaffolding for Cell Culture (Alginate+Konjac)

1. Weigh 0.1-2 g (0.1-2%), preferably about 1 g (1%) Sodium Alginate.
2. Add 100 ml water into the blender.
3. Add the Alginate powder into the blender and blend the mixture until dissolved.
4. Cover the container with plastic film and put the Alginate solution into refrigerator overnight to eliminate the gas bubbles.
5. Weigh 0.5-5 g, preferably about 3 g, Konjac and put in mold.
6. Add Alginate solution into the mold with approximately 1-15 mm, preferably about
8 mm thickness.
7. Stir the mixture until all flour dissolves.
8. Steam the mixture under low heat for 30 min until the shape is set.
9. Cover the mold with the lid and rest under room temperature for 30 min.
10. Weigh 0.1-2%, preferably about 1.5%, Calcium Lactate and stir to dissolve in water.
11. Immerse the scaffold with 0.1-2%, preferably about 1.5%, Calcium Lactate solution for at least 2.5 hours to allow formation of membrane around the scaffold.

What is claimed is:

1. A method for meat production by in vitro cell culture, comprising:
isolating tissue from an animal or plant source and making a cell suspension of cells;
increasing expression of a protein in the isolated cells by altering a level of one or more micro RNAs that regulate the expression of the protein; and
growing the cells on a food grade scaffold in a culture medium, the cells growing into a solid or semi-solid structure that mimics an animal organ.

2. The method of claim 1, wherein isolating the tissue comprises isolating organ tissue from a fish.

3. The method of claim 2, wherein the organ tissue is derived from fish swim bladder of a fish from the Osteichthyes class.

4. The method of claim 3, wherein the protein is collagen I.

5. The method of claim 4, wherein the micro RNAs are micro RNA21 (miR-21), micro RNA 133 (miR133) inhibitor or combination thereof.

6. A method for meat production by in vitro cell culture, comprising:
isolating tissue from an animal or plant source and making a cell suspension of cells;
growing the cells on a food grade scaffold in a culture medium, the cells growing into a solid or semi-solid structure that mimics an animal organ; and
co-culturing the cells with bioengineered cells that secrete nutrients, growth factors, and cytokines that support the growth of the cells.

7. The method of claim 6, wherein isolating the tissue comprises isolating organ tissue from a fish.

8. The method of claim 7, wherein the organ tissue is derived from fish swim bladder of a fish from the Osteichthyes class.
